(19) United States
(12) Patent Application Publication
(10) Pub. No.: US 2024/0062854 A1
(43) Pub. Date: Feb. 22, 2024

(54) IMPROVED CLINICIAN-CENTERED CLINICAL TRIAL VERIFICATION SYSTEM AND METHOD

(71) Applicant: Akyrian Systems LLC, New York, NY (US)

(72) Inventors: Edward Ikeguchi, New York, NY (US); Peter T. Jackson, Brooklyn, NY (US)

(73) Assignee: Akyrian Systems LLC, New York, NY (US)

(21) Appl. No.: 18/038,862

(22) PCT Filed: Dec. 7, 2021
(86) PCT No.: PCT/US21/62095

§ 371 Date: May 25, 2023

Related U.S. Application Data
(60) Provisional application No. 63/122,677, filed on Dec. 8, 2020.

(51) Int. Cl. G16H 10/20; G16H 10/60; G16H 15/00

(52) U.S. Cl. CPC G16H 10/20; G16H 10/60; G16H 15/00

(57) ABSTRACT

An improvement to a clinical trial study workflow system and method providing a method for electronic verification of a source for a source capture in a clinical trial workflow system, the method including opening an EMR system, logging into the EMR system on behalf of a user using securely stored credentials, automatically selecting correct navigation links of the EMR system and filling out the appropriate navigation forms to reach an EMIR page with desired information for a correct study subject's record and visit date, capturing one or more screenshots of one or more relevant pages containing the desired information for answering one Nor more questions on a case report form (CRF), and verifying that the one or more captured screenshots is of the correct study subject by using one or more data points for verification.

This application claims priority to U.S. Provisional Patent Application No. 63/122,677 filed Dec. 8, 2020, entitled “IMPROVED CLINICAL TRIAL VERIFICATION SYSTEM AND METHOD” and PCT Application PCT/US21/33900 filed May 24, 2021, entitled “CLINICIAN-CENTERED CLINICAL TRIAL VERIFICATION SYSTEM AND METHOD” which are hereby incorporated by reference in their entirety.

The present invention provides an improvement to a clinical trial verification system that allows a more efficient and streamlined workflow answering and entering information to clinical trial questionnaires. This improvement includes additional electronic verification that the correct patient record is being used to create the source capture. Furthermore, in addition to providing more certainty about the origin of a source capture, the present invention can also provide verification that the EMR record used to create the source capture is the correct patient and visit date.

### BACKGROUND

This idea pertains to the field of clinical research, in which experimentation is conducted upon human beings in order to assess the safety and efficacy of new medical treatments. The process of data collection for a clinical trial begins with an interaction between a physician (the Investigator) and a patient (the Subject) which is documented in writing in the patient's medical record and considered to be a legal document. The medical record consists primarily of alphanumeric characters written by the physician to describe the interaction with the patient and is considered to be the first moment in time when a record is created: this is referred to as the Source Document (SD). The physician records information in the SD about the patient's complaints, prior conditions, medications, allergies, physical examination findings, lab tests, imaging tests, and plan, for example. For purposes of clinical research, however, only a subset of the information in the SD is needed. For example, a clinical trial about hypertension might only require information about blood pressure, and even though the SD might also contain information written by the doctor about the patient's prostate exam, only the blood pressure needs to be extracted as relevant data from the SD.

Manufacturers of medical treatments will frequently recruit the help of Investigators to conduct clinical studies. These manufacturers are known as Sponsors. Sponsors will create forms (also known as a case report form or CRF) that should be filled out by the Investigators and/or their hospital staff. The CRF contains questions about the Subject relevant to the clinical trial. Answers to these questions must be submitted by the Investigator in order for the Sponsor to gather data about the efficacy and safety of their medical treatment. Upon receipt of the completed CRF, the Sponsor then transcribes information from the CRF into a database. Since this transcription process is a point for potential human error, Sponsors will often enter the data twice and later reconcile any errors of transcription. This process is known as double data entry (DDE).

It is recognized that another potential step that may lead to human error is the process in which information is transcribed by the Investigator or their staff from the SD into the CRF. In order to minimize this potential for error, Sponsors perform a process in which the information written on the CRF is checked against the information in the SD. This process is known as Source Document Verification (SDV). In practice, this requires personnel representing the Sponsor to carry the CRF to the medical office of the Investigator in order to view the SD and perform a comparison. This process is very tedious, time-consuming, and requires much traveling and cost.

Over the past two decades, the industry has adopted a new technology to enable a CRF to be presented to an Investigator in an electronic form. The electronic CRF (or eCRF) is typically a web-based form viewed via a computer with areas of text representing questions followed by any one of a variety of mechanisms to allow the Investigator to submit answers: open text fields, radio buttons, drop-down menus, etc. However, the process of filling out an online form representing an eCRF is still performed by the Investigator referencing the patient chart (SD). Therefore, this continues to be a transcription process and subject to human error. Therefore, even using eCRF, there is still a need for the Sponsor to perform quality control checks through SDV.

There are advantages to using an eCRF, however, because there is no longer a need for paper forms to be faxed between Investigator and Sponsor, and electronic forms are more legible than handwritten forms. Also, eCRF's can be programmed so that potential errors may be flagged by the eCRF at the time of data entry. For example, if an Investigator enters a heart rate value of 800, an eCRF can be programmed with computer code to detect when the entered value is out of range. Such computer code is referred to as an Edit Check. In this example, an Edit Check could be used to alert the Investigator that the heart rate of 800 is out of range. The Investigator would be able to immediately react to the problematic data and make a correction. If a paper CRF were being used, this same example would lead to many days lost in the data cleaning process for the clinical trial because the doctor might not notice the error, the paper CRF would be faxed to the Sponsor, the erroneous data would be entered into a database, and many days to weeks later, an Edit Check would be programmed and applied at the database level to flag the data as out-of-range.

These flags raised for problematic data are known as Queries. A report of this Query would then have to be printed and faxed back to the Investigator who would try to resolve the Query by referencing back to the SD. This process of cleaning data is time consuming for any clinical trial, and generally adds weeks to months, and even years of time to the drug development cycle. While the use of an eCRF can save some time in the data cleaning process, significant time and expense is still incurred in the process of programming Edit Checks. To sum, whether a Sponsor uses a paper CRF or an eCRF, there are many weeks to months spent by data management personnel to program edit checks for each question of a CRF. Also, the process of transcribing information from a SD to CRF (or eCRF) is still a point of potential human error, and therefore still requires the Sponsor to perform SDV.

One prior art process for verifying clinical trial source data is described in U.S. Pat. No. 7,669,114 to Wood which teaches software and hardware for a clinical studies system including creating new paper CRF data collection documents with unique identifiers that are organized and tracked by the unique identifiers in a local system. The CRF documents are then uploaded to a central system and reviewed to accept them as clean or to require clarifications. The original paper CRF filled out by the investigator can be represented in a digital way and transmitted via a network from the doctor's office which is on the same network with the same software installed. Using Wood, there is no longer a need to fax or mail forms to the pharmaceutical company or create “triplicate” carbon copy forms. Doctors print blank CRFs directly from the Wood system and then have a paper copy to fill out. Importantly, doctors are still filling out the paper CRF using their own handwriting. Completed forms are then scanned back into digital form and into the Wood software. The completed forms are then transmitted via the network to a “Central System” which becomes a database to keep track of documents. The Wood patent also describes scanning of separate source documents (documenting a physician-patient interaction) into the system and transmitting them to a central system in the same way as the CRF. Likewise, any data queries that are created by pharmaceutical data managers can be scanned into the Wood system and transmitted to the doctor in digital form. However, source document verification must still be performed in the Wood patent system because the doctor is still transcribing information from the source document to the paper CRF. Since the source document is scanned into the same system as the CRF, there is no longer a need to travel to a doctor's office to perform the source document verification. Pharmaceutical personnel can verify information on the CRF from anywhere there is a network connection and the Wood software system. Although the CRF is described in Wood is “digital,” it lacks the ability to accept data directly onto it and requires that it be printed to paper in order to be filled out by a physician or nurse and then scanned back into the system. In other words, while the form is electronic, the data capture is not.

U.S. Pat. Nos. 10,706,958, 10,811,122, U.S. patent application Ser. No. 16/919,396 and U.S. patent application Ser. No. 17/021,456, which are hereby incorporated by reference in their entirety, teach a clinical trial study system and method that allows an investigator to create an electronic data document (EDD) in response to a clinical trial research case report form (CRF). A snippet is created by the medical professional as part of the EDD to respond to questions on the CRF. Instead of doctors transcribing information from a patients' chart to an electronic CRF (eCRF), doctors use digital images of source documents. For example, they are asked to log into their electronic health records (EHR) (or electronic medical records (EMR)) systems and select portions of the screen that correspond with information that answers each question on the eCRF and create an image of the portion of the screen (such as a JPEG file). The workflow of the clinical trial data collection has the medical professional at the medical site capture screen images and subsequently create individual snippets for each question of the CRF. The medical professional then reviews and signs the completed form and sends to the sponsor. Once received by the pharmaceutical company, pharmaceutical data management personnel use the snippets and transcribe the information into the corresponding fields in the clinical database by accessing the unmasked parts of the image included in the EDD. The pharmaceutical companies receive image information taken directly from the doctor's official electronic medical records. Using this approach, there is usually no reason to double-check or validate answers by later reference to a Source Document (SD). However, many of the transcribed data points may later be edited to correct errors. In these cases, the medical professional must re-review and re-sign the documents when data has been changed. This may occur multiple times, which leads to inefficiencies.

Recently in the industry, there are some systems created that “integrate” with several popular EMR backend data systems through APIs and SDKs published by the EMR manufacturers. While these systems may lead to rapid acquisition of data from the EMR software, they have been slow to be adopted. This is due to several reasons which are resolved by the present invention.

One reason the existing systems are not adopted is because there is an enormous universe of EMR manufacturers, each with their own integration interface. This leads to difficulties of scale when deploying backend integration systems in clinical trials. A single clinic or hospital that uses a less popular EMR system causes the entire study to require a hybrid process.

Another reason existing systems have not been adopted is because EMR integrations require interaction with EMR vendors and hospital systems in order to set up the integration. This often requires deployment of an on-site server and construction of a VPN tunnel to securely extract data. Even if an integration was already developed with a given EMR system, which there is not, it would still require vendor and site buy-in and a deployment process with minor customization for each individual site. This is a heavy undertaking and could be blocked by bureaucratic processes or technical impediments at any individual site. The present invention overcomes this problem by requiring only user credentials comparable to a site onboarding for a typical clinical user and no vendor interaction or buy-in.

A third reason the existing systems have not been adopted is because EMR manufacturers and hospitals place little emphasis on clinical trials as a primary source of revenue. This leads to a lack of alignment in key stakeholders and little motivation on the part of the EMR manufacturers and hospitals to cooperate with integration. beyond the stock interfaces offered through their APIs or SDKs. This has led to very little advancement in this clinical research methodology over the past decade. The present invention overcomes this issue by interacting with EMRs in the same way a user does. Anything a user can do; the present invention can do. This removes the limitations imposed by EMR manufacturers on the type pf integration possible. It also removes their incentive structure on the type of integrations possible.

Pharmaceutical companies tend to keep their data transfer systems and informatics systems proprietary since they do not want research data to go easily outside of their own corporate firewalls. Thus, computer systems and informatics systems of pharmaceutical companies are not easily “plugged into” hospital systems. Thus, there exists a need to safely and confidentially transmit and verify original patient data recorded in hospital systems and to provide a convenient and reliable way that complies with patient privacy restrictions to provide necessary clinical trial data to informatics systems of pharmaceutical companies which have engaged such hospital systems to participate in clinical trials for medical treatments developed by the companies. There exists a further need to efficiently create EDDs and distribute the work required to create and verify them among the parties (clinical investigators and sponsors) in the best position to do what is required.

### SUMMARY OF THE INVENTION

The present invention provides a computerized system and method for allowing a person from the medical site, such as at least one medical professional (Investigator) to answer questions from a clinical trial questionnaire (such as an electronic case report form (eCRF)) pertinent to a clinical trial of a medical treatment by performing a source capture and delineating specific areas of the source capture image that contain an answer. The system enables a workflow, whereby an image of an answer, for example, a screenshot capture snippet, obtained through mouse clicks is used instead of transcription of alphanumeric values.

In a further embodiment, the system allows the person from the medical site, such as a medical professional user, to reorient how the questions are answered in the eCRF by allowing the medical professional to answer and organize the questions in an order best suited to a particular EMR.

In a further embodiment of the present invention, after at least one source capture (SC) is performed at the medical site and snippets created to initially answer the eCRF questions, the inventive system remembers where in a SC of an EMR page specific questions from the eCRF were answered. The inventive system will provide recommendations to question-answer pairs or create EDDs for subsequent visits selected by the medical site, in which the medical professional user will accept/confirm the inventive systems suggestions.

Another embodiment of the present invention uses Robotic Process Automation (RPA) to automate elements of existing source capture and source document verification (SDV) workflows. This includes automating the processes of accessing the EMR; locating the correct EMR document or data; extracting the EMR data in the form of both screenshots of the electronic source document and extraction of discrete data through automated copying and pasting and through directly reading data from either Windows forms (for native Windows EMR applications) or HTML Document Object Model (for web EMR applications); redacting personal identifying information (PII) through PII pattern matching, OCR, and machine learning; and transmitting the redacted SC and SDV data to centralized servers for storage, collation, analysis, and utilization. In a further embodiment of the present invention, the medical professional creates a source capture and matches CRF questions to portions of the source capture and forwards this information to a non-medical site user. The non-medical site user will create snippets and perform data entry, alleviating the work necessary from the medical site. The medical professional at the medical site reviews and signs off on the final CRF package for accuracy and does not need to review individual changes that may be made during the process.

The present invention provides a clinical trial study workflow method including a medical site professional selecting a clinical trial study, the medical site professional selecting a patient in the clinical trial study, wherein a list of visits of the patient are provided, the medical site professional selecting a specific patient visit, the medical site professional reorienting the clinical trial questions on a clinical research form to match an order of viewable information in an active operating system, the medical site professional creating a source capture of relevant information from the specific patient visit viewable on the active operating system, the medical site professional selecting a question from the clinical research form and associating the relevant information on the source capture with the question from the clinical research form, wherein the question from the clinical research form are reoriented to be answered in the order of the information on the active operating system. the medical site professional providing the associated source capture to a clinical trial management professional, the clinical trial management professional creating a snippet, the clinical trial management professional inputting information from the snippet into a clinical trial database, a second clinical trial management professional inputting information from the snippet into the clinical trial database for verification, and the medical site professional reviewing and approving complete and finalized clinical research form.

Another embodiment of the present invention provides a clinical trial study workflow method answering clinical trial questions including a medical site professional reorienting the clinical trial questions to match an order of viewable information in an active operating system, the medical site professional creating a source capture of a source document for a first patient visit, the medical site professional selecting a question from the clinical research form and associating relevant information on the source capture with a question from the clinical research form, wherein the question from the clinical research form is answered in the order of the viewable information on the active operating system, the medical site professional creating additional source captures for remaining patient visits, a system associating information from the additional source captures to related clinical trial questions creating question answer pairs, and the medical site professional approving or rejecting the system question answer pairs with an electronic signature.

Another embodiment of the present invention provides a clinical trial workflow method including a medical site professional selecting a clinical trial study, the medical site professional selecting a patient in the clinical trial study, wherein a list of visits of the patient are provided, the medical site professional selecting a specific patient visit, the medical site professional creating one or more source captures of specific patient visit information viewable on the active operating system, the medical site professional providing one or more source captures to a clinical trial management professional, the clinical trial management professional associating information on the one or more source captures with a question from the clinical research form, wherein the clinical trial management professional continues associating information on the one or more source captures until each of the questions on the clinical research form are answered, the clinical trial management professional creating snippets using the associated information and question pairs, the clinical trial management professional inputting information from the snippets into a clinical trial database, and an investigator reviewing the inputted information and approving the inputted information with an electronic signature.

Another embodiment of the present invention provides a clinical trial workflow method using robotic process automation, the method including opening an EMR system, logging into the EMR system on behalf of the user using securely stored credentials, automatically selecting correct navigation links of the EMR system and filling out the appropriate navigation forms to reach an EMR page with desired information for a correct study subject's record and visit date, capturing one or more screenshots of one or more relevant pages containing answers to one or more questions on a case report form (CRF), automatically marking the one or more captured screenshots corresponding to an area that contains answers to individual questions on the CRF to create a snippet, permanently redacting the areas with personal identifying information before the one or more screenshots leaves the robotic process automation system, wherein the robotic process automation system identifies a portion of the one or more captured screenshots containing the personal identifying information using optical character recognition and a mapping of discrete text data read from the EMR page, identifying sections of the one or more captured screenshots containing answers to one or more CRF questions using OCR and a mapping of discrete text data read from the EMR page by the RPA system to ascertain alphanumeric data represented by the one or more captured screenshots, offer suggestions for data entry using the identified sections, formulating an electronic data document (EDD) by combining and associating the snippet with the one or more captured screenshots, the one or more questions from the CRF, the alphanumeric data represented in the snippet, and a full audit trail of actions, transmitting the EDD to a server over a secure network, automatically selecting a logout button on the EMR system for the user and providing the user with a report summarizing the actions taken by the RPA system and the EDD for review, commenting and/or editing.

In a further embodiment, a device and method for adding additional security and further reduces the risk of inadvertent error using the inventive system. This verification may be driven by OCR and programming logic, secure and encrypted communication (a “handshake”) between the inventive software and the EMR system, checking active EMR application windows, Inter-Process Communication (IPC) and web-based API's.

Another embodiment of the invention provides an improvement to a clinical trial study workflow system and method providing a method for electronic verification of a source for a source capture in a clinical trial workflow system, the method including opening an EMR system, logging into the EMR system on behalf of a user using securely stored credentials, automatically selecting correct navigation links of the EMR system and filling out the appropriate navigation forms to reach an EMR page with desired information for a correct study subject's record and visit date, capturing one or more screenshots of one or more relevant pages containing the desired information for answering one or more questions on a case report form (CRF), and verifying that the one or more captured screenshots is of the correct study subject by using one or more data points for verification. The verification may occur with real-time scanning by the clinical trial system of the one or more screenshot captures to detect the presence of keywords of the one or more data points, and assessing if the one or more captured screenshots is verified or unverified, wherein the one or more captured screenshots is labeled verified if a defined number of the one or more data points is positively identified in the one or more captured screenshots by the clinical trial system, wherein the one or more captured screenshots is labeled unverified if the defined number of the one or more data points are not positively identified by the clinical trial system. The verification may also occur wherein the electronic verification includes a secure and encrypted two-way communication including a handshake when a connection is established and verified to be the EMR system providing the source for the one or more captured screenshots, and once the source is verified, requesting the EMR system to confirm it is displaying information about the correct study subject.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is an exemplary screenshot of the user study selection page;

FIG. 2 is an exemplary screenshot of the landing page for the user who will perform source capture;

FIG. 3 is an exemplary screenshot for a medical professional user showing a patient's visit status;

FIG. 4 is an exemplary screenshot showing CRF questions on the SC performer's landing page;

FIG. 5 is an exemplary screenshot for the SC performer's showing CRF question tabs;

FIG. 6 is an exemplary screenshot for the SC performer showing an application selection window;

FIG. 7 is an exemplary screenshot showing a patient record open in an EMR;

FIG. 8 is an exemplary screenshot showing redacted information on a SC;

FIG. 9 is an exemplary screenshot showing the inventive system overlaying existing applications;

FIG. 10 is an exemplary screenshot for a SC performer showing a SC HUD;

FIG. 11 is an exemplary screenshot showing snippets created using markup palette;

FIG. 12 is an exemplary screenshot showing snippets on a SC in form view;

FIG. 13 is an exemplary screenshot showing a CRF progress screen for the SC performer;

FIG. 14 is an exemplary screenshot showing a landing page for a data entry user;

FIG. 15 is an exemplary screenshot showing a data entry user landing page for backup data entry with a corroborator;

FIG. 16 is an exemplary screenshot showing a landing page for the user that reviews completed CRF packages;

FIG. 17 is a flowchart of an example workflow 1;

FIG. 18 is a flowchart of example workflow 2 with data entry performed by the monitor;

FIG. 19 is a flowchart of example workflow 3 with data entry performed by a data manager;

FIG. 20 is a flowchart of example workflow 3.

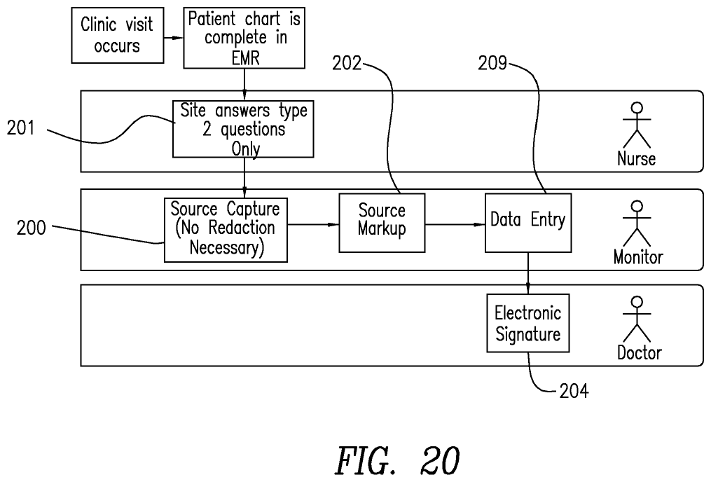

FIG. 21 is a flowchart showing an exemplary embodiment of the process of SC verification with an EMR in the present invention; and

FIG. 22 is a diagram showing another exemplary embodiment of the process of SC verification with an EMR system in accordance with the present invention.

### DESCRIPTION OF THE INVENTION

This invention provides a computerized system and a method allowing Investigators (doctors, nurses or other designated medical professionals) to provide SCs to Sponsors corresponding to the answers to questions posed by a CRF or an eCRF. SD image sections (snippets) of the SC are then prepared in question-answer pairs to address each question on a CRF or eCRF. Snippets may be digital photographs or an output of an imaging device, where the imaging device may be a digital camera, computer screen capture software, a mobile telephone, a medical scanner or imaging machine and a portable computer. A snippet also includes direct electronic or visual recordings extracting audio or visual segments or both (not metadata). This would include a direct electronic recording from telemedicine interactions, for example. Since the eCRF questions are being answered by images coming directly from the SD, it is no longer a transcription process, and therefore obviates the need to perform SDV. Also, since the SD is the ultimate resource of original data, there is virtually no need for programming edit checks to evaluate the quality of the data or to do other time-consuming data cleaning. The SD is considered to be a legal document and the information in it is considered to be final. Also, in modern times, most doctor's offices and hospitals have adopted electronic medical records (EMR) and these systems already contain complex error-checking programming. This invention enables Sponsors to leverage the error-checking that already exists in EMR systems.

The current idea presents a process in which the Sponsor is able to perform single or double data entry directly from relevant portions of the SD, thus skipping the step where the Investigator fills out the CRF with alphanumeric data. The invention involves a method in which the Investigator fills out the CRF with SC of all or select portions of the SD where the relevant alphanumeric information is visible. SD images can be obtained by a digital camera for paper source documents or the screen-capture capability within software for electronic SD. In order to protect patient privacy as required by the Health Insurance Portability and Accountability Act (HIPAA), FDA Title 21 CFR Part 11, and other laws and regulations, and to direct the Sponsor's attention to only the relevant information, the Investigator is able to mark the images with drawing and labeling tools. Also, to protect patient privacy, areas that are not marked by the Investigator are made invisible to the sponsor by redaction (automatic or manual) such that the sponsor may see only the alphanumeric information on the SC that the Investigator decides are relevant and responsive to the clinical trial.

Since the Investigator is no longer filling out a form, but rather, taking SC from the original SD, there is no longer a risk for transcription error. Since the Sponsor is viewing an exact digital image of the section of the SD needed for the clinical study, single or double data entry is performed directly from an image of the SD, and therefore eliminates or greatly reduces the need to perform in person SD verification.

The present invention may be implemented by a medical professional at the medical site, such as a physician or nurse, taking a SC of a page (for example, a screen shot or a snap shot of a particular section on a page), labeling that SC to identify the relevant data it contains (e.g., temperature, blood pressure, etc.), and adding any comments relating to the data or its application to a clinical study to that SC. Thus, without having to enter patient information into a new form, the medical professional can identify information generally relevant to studies that might be performed by various pharmaceutical companies. Similarly, a medical professional may take a photo (e.g., with a smart phone app) of paper test records unavailable on a computer, label that information with or without comment, and electronically include such test records using the same program. The patient information that is not associated to a CRF question by the medical professional is electronically redacted, thus allowing only study-relevant material to be shared electronically with the pharmaceutical company. The company receives this information in a manner such that only the portions of the record that are selected by the medical office are shown, either surrounded by redacted portions of the SC or alternatively showing just the relevant data in each source capture, one by one. This, in turn, shows the pharmaceutical company all the data it needs to see and none of the information that is irrelevant to the study and/or raises privacy issues. Because the sections of a medical record are selected and labeled by the medical office, the pharmaceutical company can determine what information it wants to see on a study-by-study basis using a Clinical Research Form (CRF) and the present invention will provide that information. This will save the pharmaceutical company time in having to review an entire medical file to verify the information. And the information can be shared electronically without raising privacy concerns because only study-relevant information can be viewed by the Sponsor's reviewers.

Furthermore, if redacted portions of the medical file need to be reviewed for regulatory or fraud elimination purposes (e.g., by an FDA auditor, or a high-level executive of the Sponsor company), a user with the proper authorization is able to view the redacted information in a limited but sufficient manner (e.g., a viewport). Thus, the invention masks the confidential information from general view but retains the data of that information as part of the redacted SC in case an authorized person needs to view it to, e.g., confirm that the data being shared is in fact related to a particular patient.

The present invention provides a system with multiple user interfaces including an interface to perform SC, an interface to perform data entry and an interface to review completed CRF packages for approval. The use of different interfaces provides personalization to the specific roles to be performed by the user. For example, a user preparing SC, such as a medical professional, may convert eCRF questions to a format that follows the EMR, allowing the medical professional user to answer the eCRF questions more easily and in a quicker fashion. The present invention is agnostic to the order in which questions appear on a CRF. This allows the site users to organize the order in which they answer the CRF questions to match the order the data appears in the electronic health record (EHR). The lack of synchronization, between an EHR's position of data on a page and the order in which questions appear on a CRF in prior art, leads to a great deal of ‘page flipping’ when site users respond to CRF questions using traditional methods. Using an OCR matching process the inventive system searches the full text of the SC for every possible search term configured for every CRF question, provided the key word has not already been assigned elsewhere (i.e.—there is only one location in the EHR per search term per patient visit record.) This allows the user to capture SC of SD and match the relevant locations of answers to CRF questions without any constraints around the order in which questions are presented on the CRF.

The inventive system also provides the ability to deviate from the traditional clinical study workflow and roles. Although the inventive system supports the traditional roles, it optimizes the workflow by minimizing the workload of the medical site. For example, a medical professional at the medical site will attach a source capture (“SC”) of the EMR to answer every question on an eCRF list. This SC may be individual portions of the EMR or a whole page of the EMR record which contains the question-answer associations. Personal patient information on the SC of the EMR is redacted, so the patient's information on the EMR is anonymous on the SC. Once the medical professional creates the SCs and question-answer associations, the medical professional user may be finished with the eCRF questionnaire and non-medical site personnel such as the sponsor or a third party, takes the associated questions and SCs to prepare snippets containing questions-answer pairs to the eCRF questions. To further preserve confidentiality, the sponsor may use a third party to create the question-answer snippets and deliver the snippets to the party performing data entry, such as the sponsor or monitor. Data entry is then performed using the prepared snippets. This optimized workflow shifts the majority of the work away, including the clerical work, from the medical site. (The medical site may also prepare the snippets instead of a non-medical site personnel, depending on the workflow chosen). This is a significant departure from current industry practice as the medical professional has always been the user tasked with submitting the response to the CRF question. However, since the source capture is the ultimate reference of truth, there is no longer a medical interpretation of a response required, and only reference to a location on a source document, which can be performed by the machine, affirmed by the medical user, and procedurally executed by a non-site user.

In a further embodiment, the eCRF form and questions may be programmed in the inventive system software with keywords associated with each question to allow the inventive system to scan the SC provided by the medical professional to select and group the patient records and relevant information needed. The inventive system allows a user to configure a database that will incorporate key search terms related to any given question on a CRF. When the user takes a SC of a SD, the inventive system applies optical character recognition (OCR) technology to identify matches between the content of the SC and the search terms configured for the many questions on the CRF. If a match is identified, the area(s) of the SC are presented to the user as corresponding to the search term for a particular question. A trained medical professional user who is familiar with the content of a medical chart can determine whether the area identified by the OCR process in the inventive system is correctly or incorrectly correlating to the associated question which is also shown to the user. If the correlation is accurate, the user accepts the match. If the correlation is not correct, the user can reject the area matched via OCR. In a preferred embodiment, this process of ‘CRF question-to-source capture matching’ enables the user to affirm the location where snippets should be created, but not have to create the snippet themselves. The SC can then be sent to a non-site user (such as a CRO user or Sponsor user in data management or clinical operations) to view the SC, with specific areas indicated as matches, to perform the snippeting process. (A CRO is a contract research organization employed by sponsors to conduct operational aspects of a clinical trial such as data management, site management, monitoring, etc.) Completed snippets are then used for alphanumeric data entry into a database by the non-medical site user.

In a further embodiment, the inventive system creates an EDD for all subsequent visits once the medical professional. has provided an initial SC with question-answer associations for the initial patient visit. This is done through a combination of OCR (to identify key words from the CRF on the EMR page), pattern recognition and artificial intelligence (AI). Since EMR pages are structured similarly from one visit to another, the inventive system will recognize patterns in the SC to provide suggestions for an answer to a CRF question based on the location of the answer in prior submissions.

Source Capture Interface

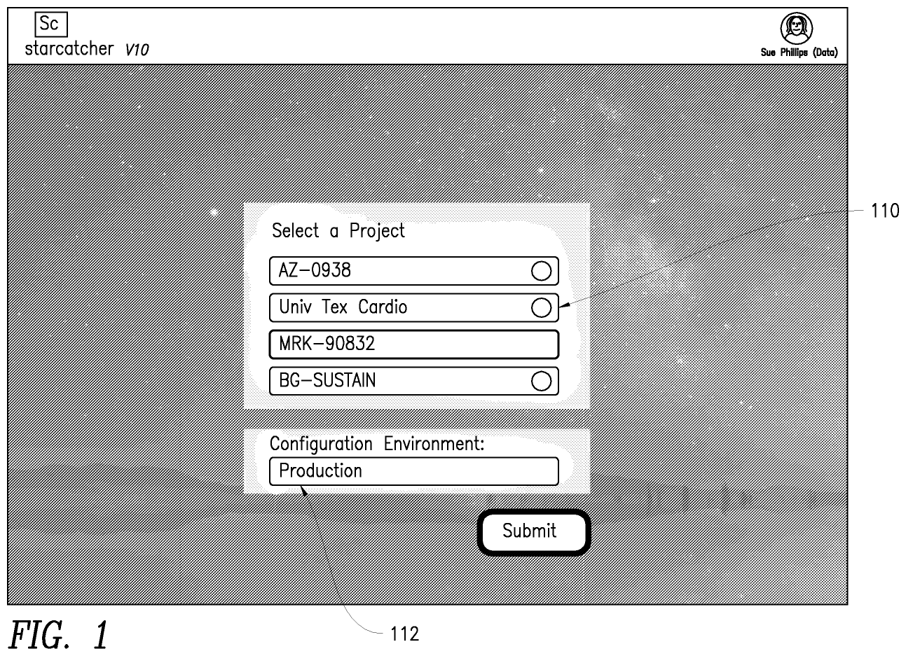

Source Capture is generally performed by someone at the medical site, for example, a doctor, nurse or other medical professional at the medical site. Medical professional user is used below to describe this interface; however, this interface can be used by any user delegated to perform source capture. Referring to FIG. 1, in an exemplary embodiment, the present invention provides a study selection screen in which the medical professional user selects a clinical trial study from menu 110 that provides a list of all the clinical trial studies available to the signed in user based on the user's access detail. The medical professional user also can select the configuration environment from menu 112 based on the user's project access detail.

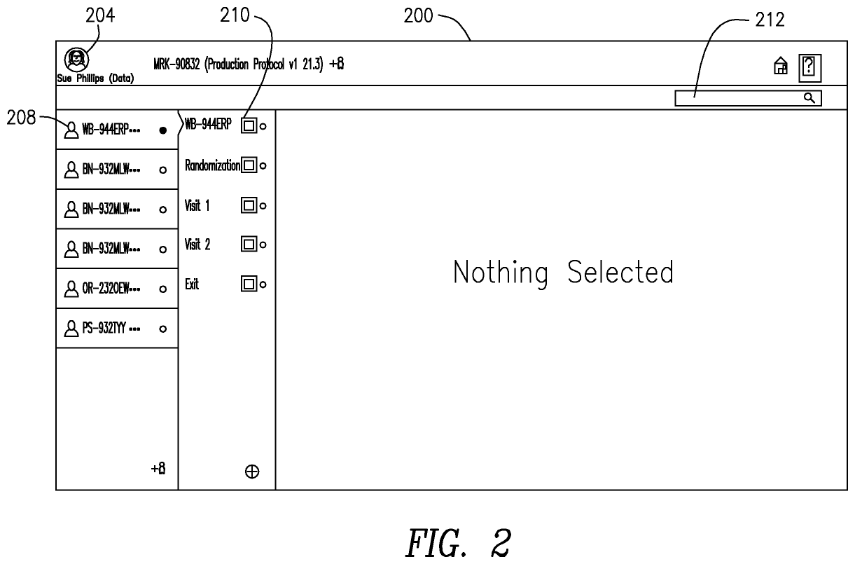

Referring to FIG. 2, in an exemplary embodiment, the present invention provides a user's landing page 200 in a grid view. Other views are available, such as a list view. Landing page 200 is specific and tailored to the user's role. The medical professional user's information 204 is provided at the top of landing page 200. Landing page 200 provides a list of all of the medical sites clinical trial patients 208 (in the selected clinical trial) and a list of all of the patient's visits, 210. Patient list 208 provides the patient's unique study ID (or patient initials) and a status indicator (color indication red/green/yellow, for example) of the progress made regarding the completion of the CRF questionnaire. Patient list 208 is sortable by patient name or status color. Visit list 210 provides a list of the selected patient's visits by name and can be filtered by visit status, such as completed visits, canceled visits, past due visits and visits with outstanding problems, for example. The user can select a specific patient and then select a specific visit to gather information from that visit. The necessary information is then selected to create a SC, associate the information to a question, and forwarded to the relevant party, such as a monitor to create a question-answer pair snippet. This is an optimized workflow where the medical sites perform SC only and the monitor or third party perform snippet creation and data entry, etc. Additional workflows may also be followed in which the medical professional does both source capturing as well as snippet creation. Search bar 212 is provided on landing page 200 that allows alphanumeric entry to search for patients, visits, forms, and questions for added convenience for the medical professional. New patients in the study may also be added.

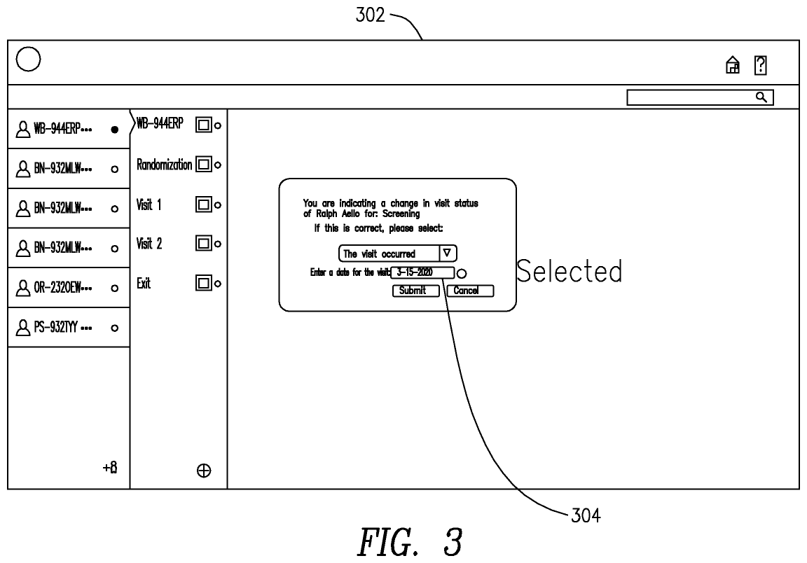

Referring to FIG. 3, in an exemplary embodiment, the present invention provides a visit status 302 of a patient. Visit status 302 may also include canceled or missed visits, for example, with reasons for such actions. The user can confirm a visit occurred and provide the relevant date 304. A calendar widget may be used to remove data entry ambiguity. This date will be the date check for all SC related to the visit. Ad hoc visits, unscheduled visits, may be added to the system by drawing from the collection of forms or data points that have already been setup for a particular clinical trial.

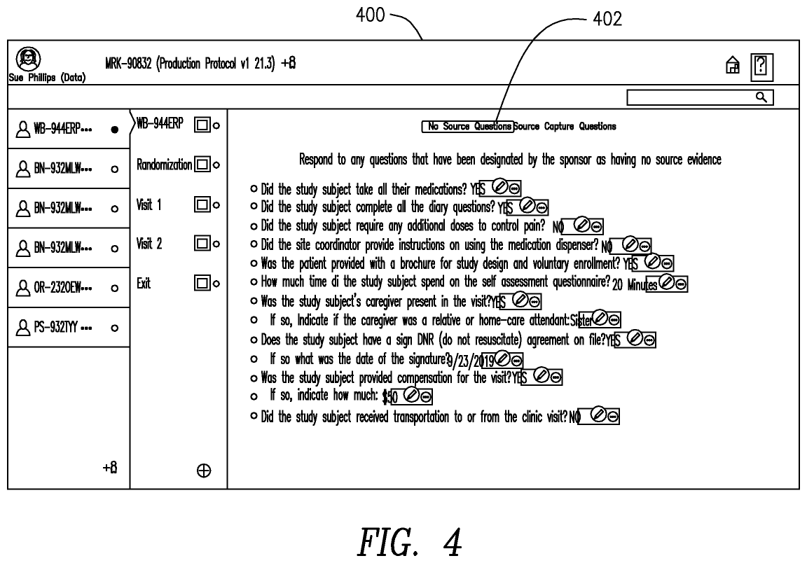

Referring to FIG. 4, an exemplary embodiment is shown with CRF questions on the SC performer's landing page displayed once the user indicates that a visit has occurred, for example the screening visit. The CRF forms will automatically populate and provide clinical trial questions on landing page 400. The user can select the type of clinical trial question they wish to answer by tabs 402—“No Source” questions or “Source Capture” questions. Based on the tab selected, the relevant questions will appear on the screen. “No Source” questions are straightforward questions that do not require source evidence to answer. They are listed and can be answered by the user with a response widget such as yes or no, or comment capability provided. “Source Capture” questions require source evidence in which the medical professional user will need to associate a SC to each CRF questions listed.

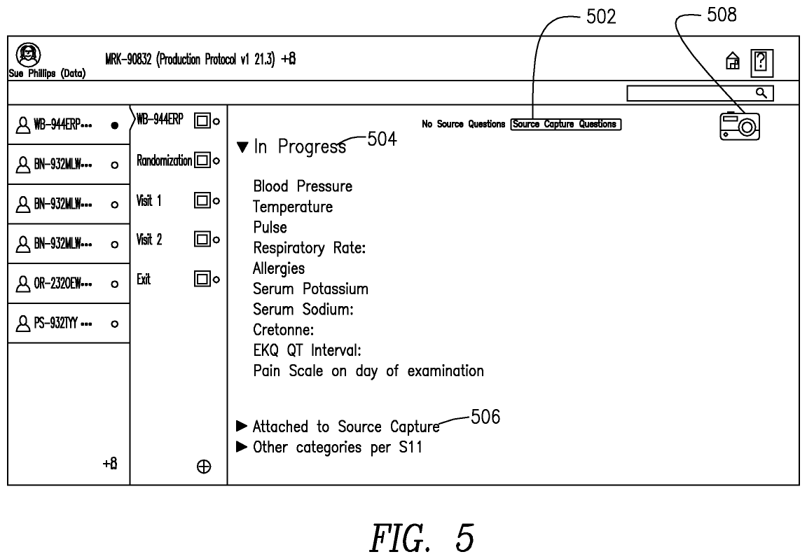

Referring to FIG. 5, in an exemplary embodiment, the present invention provides a source capture tab 502. These questions are organized into two groupings—questions that have been attached to a SC, “Attached to Source Capture” 506, and questions that have not been attached to a SC, “In Progress” 504. The user must associate all or a portion of a SC to every question on the list. Once all the questions have a SC attached, the user's job answering the clinical trial questions may be completed, depending on the chosen workflow. FIG. 5 shows that blood pressure, temperature, pulse, respiratory rate, allergies, serum potassium, serum sodium, creatinine, EKQ QT interval and pain scale are questions that are still in progress and do not have a SC attached. (These questions are only an example of what may be asked). Using icon 508, the user may begin the process to add a SC.

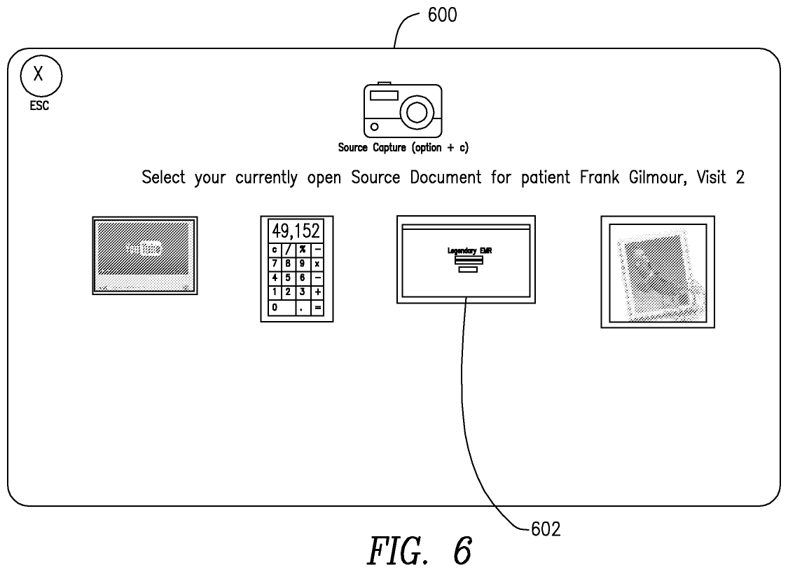

Referring to FIG. 6, in an exemplary embodiment, the present invention provides an application selection window 600. Once the user selects the source capture icon 508, application selection window 600 will open All software programs open in the operating system will appear as an icon on screen 600. The user may select and navigate within various selected applications to open the relevant record for the SC. Once the user selects the software program they wish to use, for example the EMR application 602, the user interface of the present invention becomes minimized to an adaptable transparent floating widget that remains on top of the open active application.

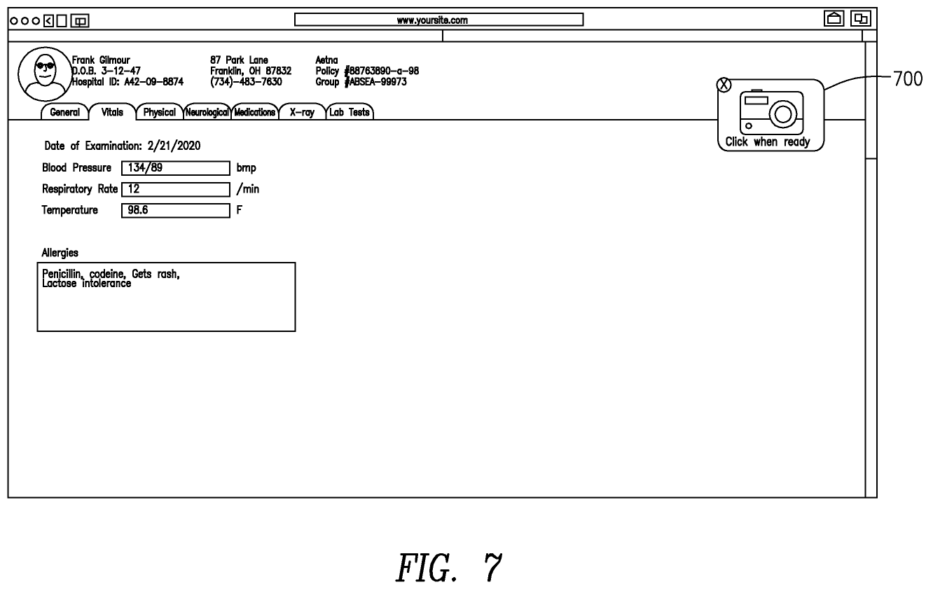

Referring to FIG. 7, in an exemplary embodiment, an open patient record in the EMR is shown. Icon 700 is the inventive system floating transparency over the active EMR application.

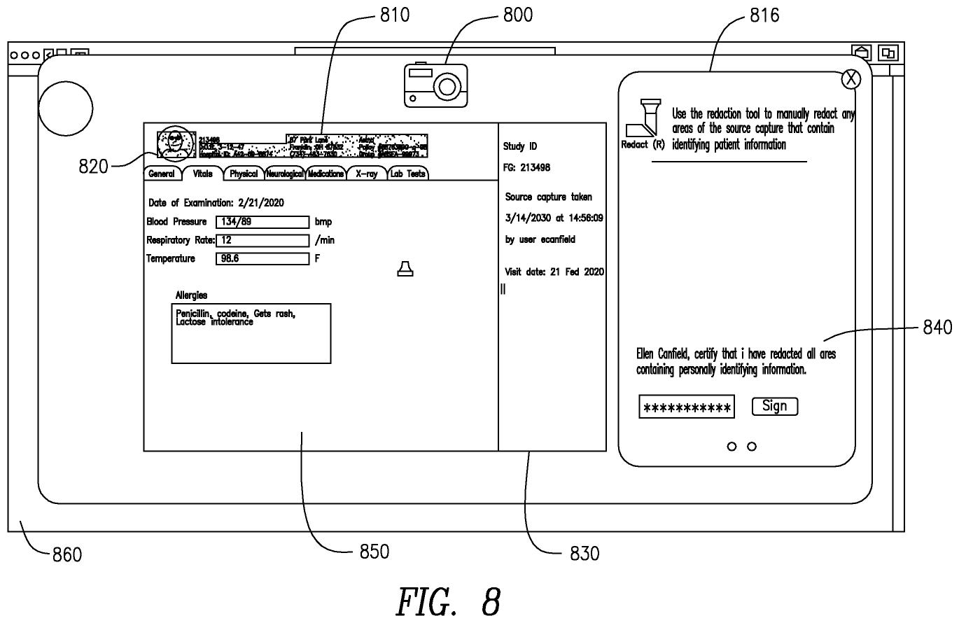

Referring to FIG. 8, in an exemplary embodiment, the present invention provides automatic and manual redaction of information and redaction certification with an electronic signature of a SC. Patient's personal information 810 is automatically redacted from SC 850. This auto redaction of personal information occurs on the medical professional's machine within a Google chrome addon for example, or at the server, such as Amazon Wed Service. On the client-side, the patient's personal information is not transmitted on a SC over a server/the internet for redaction processing. (Certification of the patient associated with the SC occurs by the machine and is then redacted prior to transmission of the SC over the network). On the server side, OCR and redaction is fast and uses the computing power of a server architecture to do the heavy lifting in terms of computation. A screenshot is taken, and the image is securely transferred to the server in fully unredacted but encrypted form. Once the screenshot reaches the server, all OCR and redaction processing takes place using ethereal memory such as RAM, and the PII is not recorded to permanent storage. The user may select between the client-side and server-side OCR and redaction using a toggle selection for example. SC 850 is located within the inventive system pane 800 which overlays the open active system application 860, for example the EMR system. Auto redaction may use Optical Character Recognition (OCR), for example, to find matches for redaction fields such as date of birth, social security number, address, phone number, email address, hospital ID, insurance carrier, insurance ID and sections of a patient ID photograph. The redacted information is blocked from non-authorized users with an overlay which may be a dark block. The user may also use redaction tool 816 to manually redact information that was not automatically redacted. For example, patient photo 820 is manually redacted with redaction tool 816 by selecting the information and surrounding it with a box. Manually redacted areas will show an “X” icon over the section when hovering over it. After redactions are made, the user confirms the redactions, both automatic and manual, using electronic signature 840. Manual redaction needs to be applied to each SC individually. A professional at the medical site, generally performs manual redactions, however a monitor for example, may also perform manual redaction of a SC.

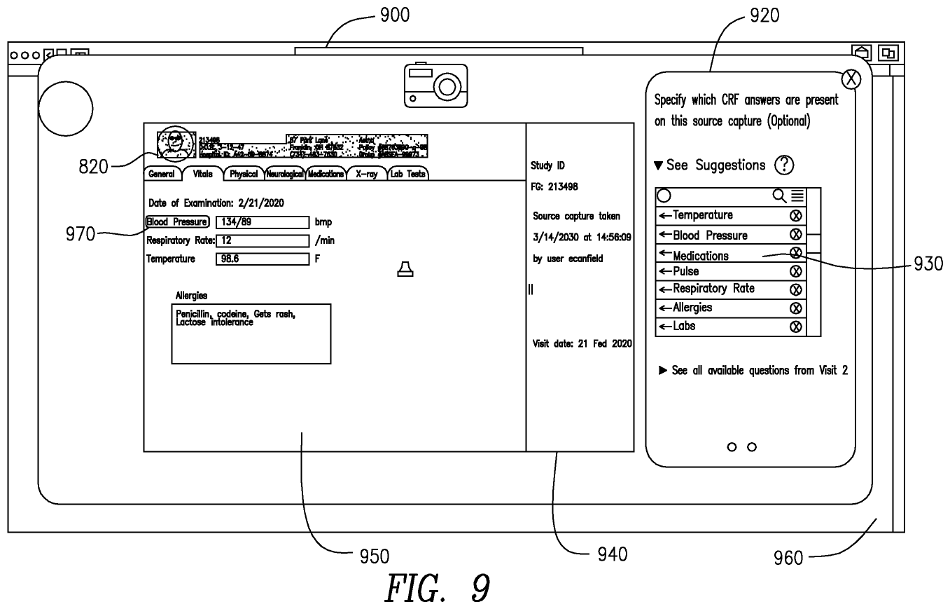

Referring to FIG. 9, in an exemplary embodiment, the present invention provides a pane 900 overlaying the existing application 960 on the system. Pane 900 includes SC 950 and SC markup window 920. Markup window 920 lists the patients study ID as well as the visit name and date. Markup window 920 also includes a question list 930, listing all of the questions for the named visit that require SC and have not been paired with a SC response. The user can select a suggested list of questions that may have answers found within SC 950 or a list of all study questions that require a SC. When the suggestion list is selected, OCR may be used to search for keywords in SC 950 that may answer a CRF question. (These keywords for search purposes may be set up during the study configuration). For example, question list 930 provides a list of questions that may have supportive evidence found within SC 950, such as temperature, blood pressure, medications, pulse, respiratory rate, allergies and labs. Hovering over/Highlighting a specific question on list 930 highlights the corresponding sections on SC 950 that contain suggested key words for that question, for example blood pressure 970. This helps the user quickly find an answer to a CRF question. Clicking the arrow next to the suggested question adds the node to the pull-out drawer and places it a SC question Heads up Display (HUD) 940. HUD 940 is a semi-transparent drawer overlaying SC 950. HUD 940 is permanently linked to SC 950. Once a question is added to SC HUD 940, it is removed from the question list 930. The user may also remove a question from list 930 by clicking the “x” if supportive evidence is not on SC 950. The user may also move a question from SC HUD 940 back to SC question list 930 by dragging it.

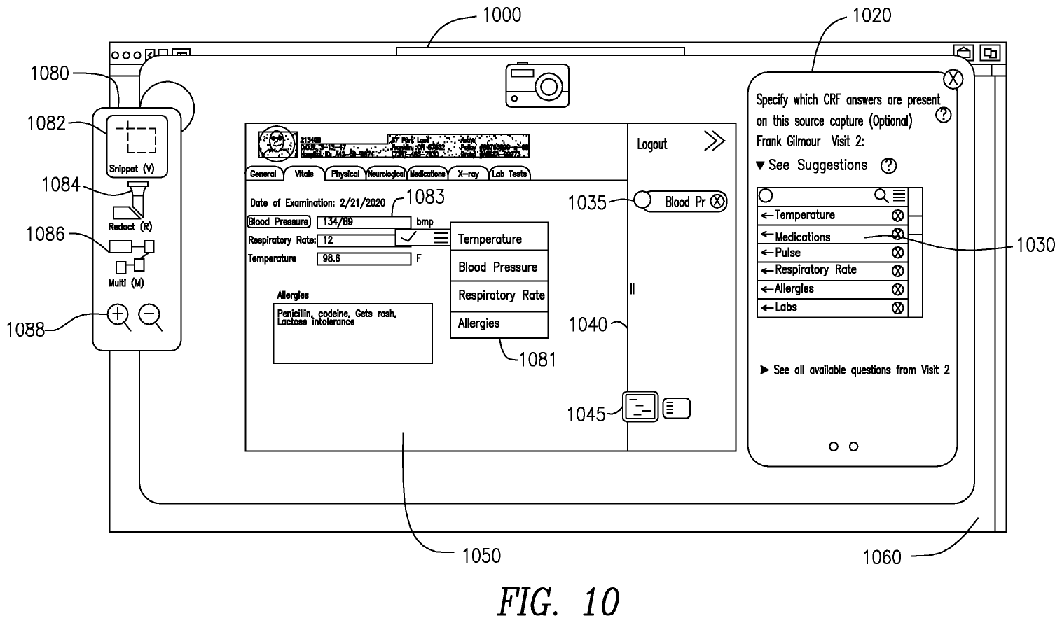

Referring to FIG. 10, in an exemplary embodiment, the present invention provides a SC HUD 1040 and computer software tools used to annotate SC. Once a question 1035 is associated with SC 1050 and added to SC HUD 1040, question 1035 is removed from list 1030, as it can only occur once, and SC Markup Palette 1080 appears. Markup Palette 1080 contains the tools relevant to the functionality available to the user's role. (For example, the data entry interface may also include the markup tool for snippet creation. User roles are established in an administrative module of the software). Markup Palette 1080 includes snippet tool 1082, redaction tool 1084, multi-selection tool 1086 and zoom tool 1088. Markup Palette 1080 may be a floating square graphic that is moveable and can be repositioned. Redaction tool 1084, as discussed above, allows the users to manually select information to be redacted. Multi-selection tool 1086 allows the user to select multiple portions of information on the SC that are not vertically or horizontally aligned by drawing a markup box around the desired information and then drawing another box from the edge of the previously drawn markup. Each subsequent area created with multi-selection tool 1086 is attached to its previous selection with a connector line. Zoom tool 1088 allows the user to zoom in or out of the SC image 1050 only. Snippet tool 1082 allows the user to create a snippet by drawing a markup square 1083 around the desired information for the snippet. It allows the user to delineate what sections of the screen capture represent evidence of the answers to specific questions on the clinical trial questionnaire. The result is one or more subsections of the larger screen capture, each representing a specific answer to the clinical trial question. Once markup square 1083 is complete, labeling tool 1081 provides a dropdown list of all questions in question list 1030. The user may select the relevant question for which the markup toolbox 1083 is associated. This freezes the markup square in place with a green border and changes the status indicator node to green, notifying the user (or other viewers) this question is answered/completed, and a snippet has been created. These smaller screen capture sections with associated answers are snippets. (Depending on the workflow chosen, the snippet creation may be done by a non-medical site user, such as a monitor, instead of the medical site user as discussed above). The snippet includes a color-coded box with an edit option and a comment box to notify the sponsor of any additional information. This snippet creation process can be repeated until all of the information on the SC that is relevant support evidence to answer a CRF question is paired to the related question. The snippets created may be edited later by the user. However, once a snippet is approved by the sponsor, it is locked and can no longer be edited. A lock symbol may be used to notify the medical professional user (or other snippet creator) that the snippet can no longer be edited.

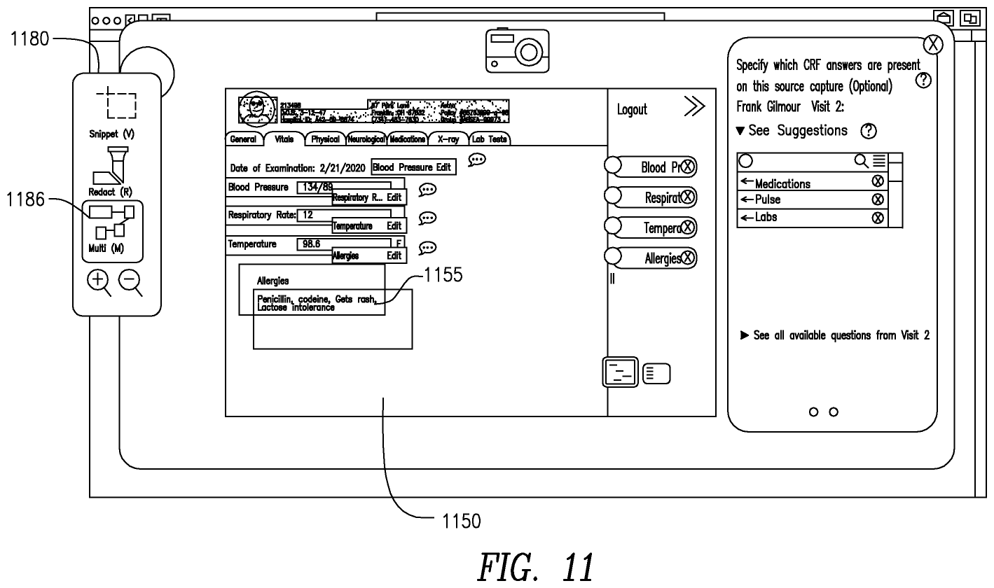

Referring to FIG. 11, in an exemplary embodiment, the present invention provides snippets 1155 created using markup palette 1180. The process is repeated for each new SC page. Status symbols are provided to notify the medical professional user when the questions have been answered for each visit for each patient. Status symbols are context sensitive depending on the user's role. If their role is to take source captures and assign CRF questions to those SCs, then the status marker will display green when their SC tasks are completed. If a user is assigned a role that requires markup of source captures to create snippets, then their status symbols will display red for any question until they have created a snippet-question pair for the question. For users configured with both source capture and snippet creation, the status symbols will remain red until SC are created and associated with CRF questions and each CRF question subsequently has a snippet-question pair.

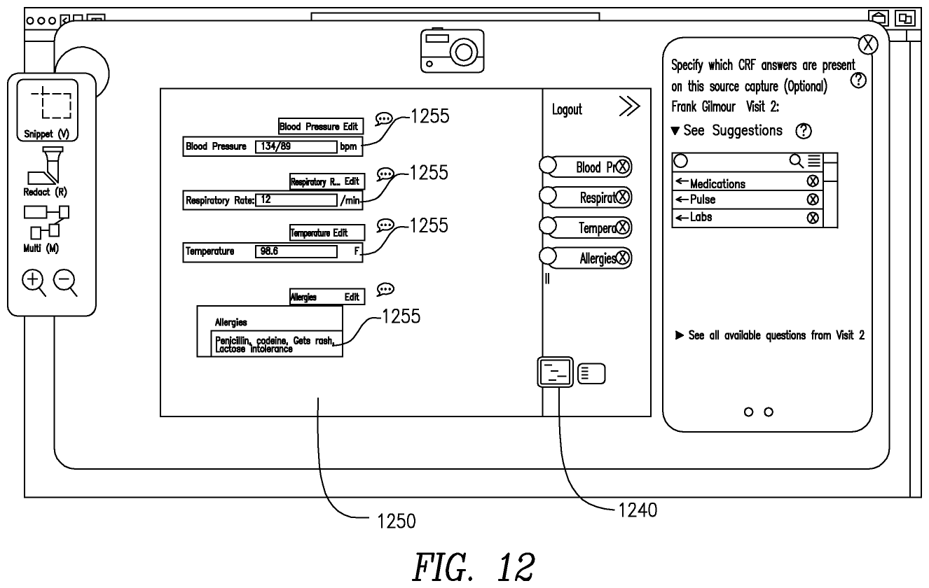

Referring to FIG. 12, in an exemplary embodiment, snippets 1255 on SC 1250 are shown in the form view. Form view shows only the snippets 1255 created from the SC 1250 and paired with a question. Other information that was on the SC but not paired with a question is not shown. Native view is the other viewing option.

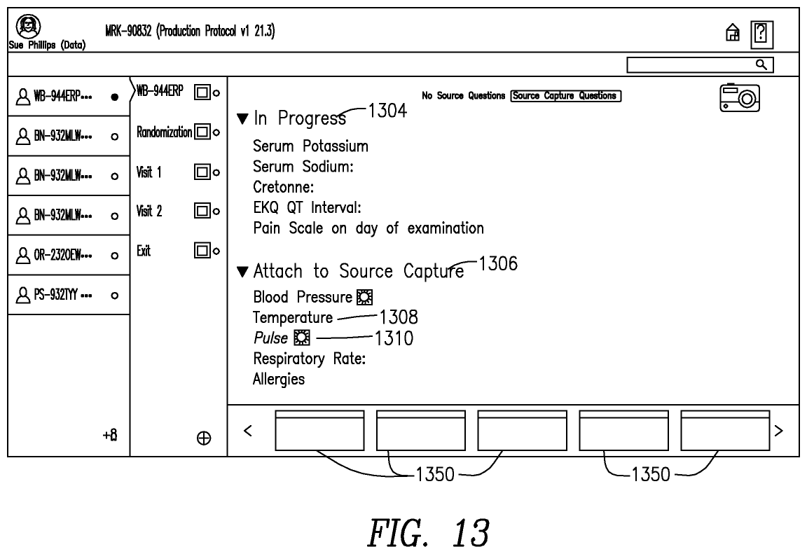

Referring to FIG. 13, in an exemplary embodiment, once the user has completed SC or selects to exit the SC process, a progress screen is provided. Questions listed “In Progress” 1304 still need SC associated with them. (For users who also have snippet/markup ability assigned to their role, a similar progress screen is provided for CRF questions that have snippets pairs or still need a snippet association.) The user may provide notes to the unanswered questions in progress such as: data unavailable, source unavailable, set a reminder or open a query. The questions under “Attached to Source Capture” 1306 have been answered using a SC. (Snippets will be created from the SC attached to each question). The system will notify the user if there are any open queries associated with an answer by placing an alert icon 1308 next to the question. Asterisk 1310 next to a question, indicates the presence of an audit trail. The audit trail provides a history of all actions performed such as responses provided, data entered, data changes, SC images marked up, etc. with the time/date detail stamps, reasons for changes and the user who performed the action. Once non-medical site personnel, such as a monitor or data manager, accepts a snippet, the question is italicized, for example, “Pulse” in FIG. 13, and locked to the medical professional user from further editing. At the bottom of the screen, a tray of all of the SC used 1350 to answer the listed CRF questions are available to the medical professional user. Hoovering over a SC will highlight the relevant questions that were answered from the highlighted SC page.

Data Entry Interface

Data entry can be performed by any party. This includes medical site or non-medical site personnel, such as a nurse, monitor, data entry manager, third party or doctor, etc. The sponsor may delegate a clinical trial management professional to perform data entry. Similar to the Source capture interface, a user on the data entry interface logs into the inventive system and selects the study and configuration environment they would like to work on.

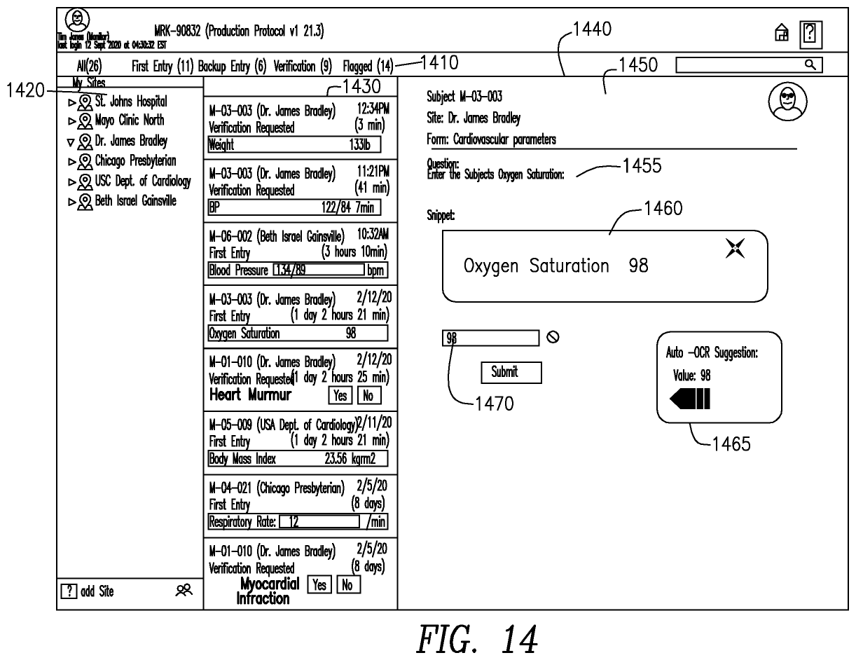

Referring to FIG. 14, in an exemplary embodiment, the present invention provides a landing page 1400 for the data entry user. The screen is specific to the data entry user's role and the selected study and environment. The data entry user may receive SC with question-answer pair snippets from the medical site user. The snippets are used to provide the information for data entry. However, if the data entry user is also creating a snippet in addition to data entry, the data entry user will receive SCs having questions associated with the SCs created by SC user. The data entry user will use this information for snippet creation and data entry. Markup palette, as discussed above, is available to the data entry user including the snippet creation tool if the user's role is to create snippets. If the same data entry user is creating both the snippets and data entry, they will be able to create snippets and enter data at the same time. The present invention allows the data entry user to organize the snippets or EDDs based on the medical sites they are responsible for. The user can select which data points they want to look at based on the data entry stage or select all to see all of the currently outstanding items. Sites column 1420 provides a list of the sites for the user and data entry specific team members. Once the data entry user selects a site, data points column 1430 opens a list of data points. These data points may be filtered using filter presets 1410 which filters the data points based on the stage of the data entry, such as first entry, backup entry, verification or flagged. Once a data point is selected from the data point column 1420, the details of the data point are displayed in data processing detail panel 1440. Panel 1440 may include data processing page header 1450, CRF question 1455, snippet boxes 1460, data entry widget 1470 and an Auto-OCR suggestion 1465. Data processing page header 1450 provides information regarding the data point. This information may include the subject ID, site name, site ID, CRF name, the nature of the request, for example, back-up entry request, and the time of the last processing step. Auto-OCR suggestion 1465 provides data entry suggestions based on the associated snippet. As a first data entry user, the data entry user may need to accept or reject the snippet, if another monitor (a non-medical site personnel) has not already done so. The data entry user will accept the SD snippet if it is supportive evidence for and representative of the answers to the questions. The data entry user will reject the SD snippet if it is not supportive evidence and is not representative of the answer to the questions. (If the snippet is rejected, the data point is processed for query placement). A data entry user enters the information from the accepted snippet box 1460 into data entry widget 1470. Once the data entry is complete, the user submits the information and the inventive system checks to confirm information has been entered, commits the information to the audit trail, and changes the data to read only text. The data entry information may still be edited at this point. If data is missing, the system will provide an error unless the site has indicated that the data does not exist for this data point. This may be set up when the system is configured.

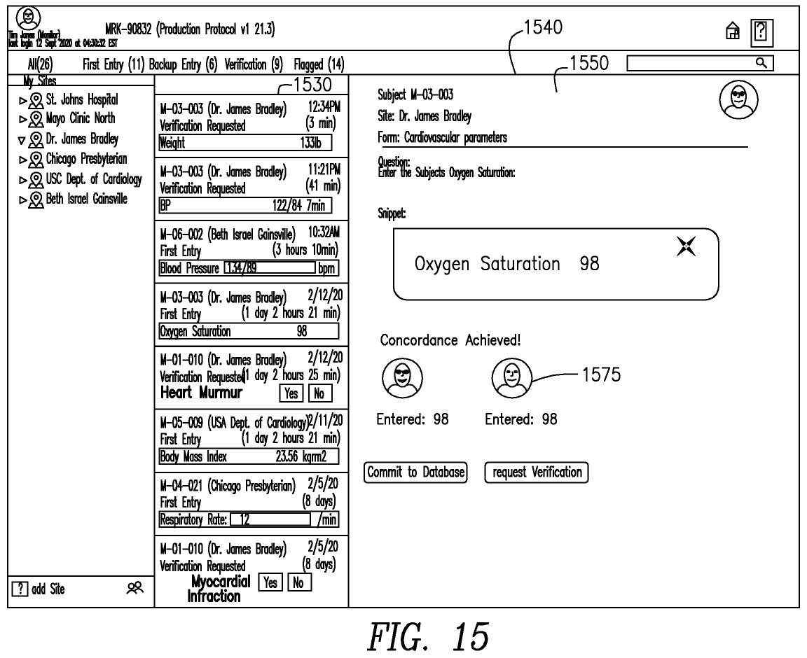

Referring to FIG. 15, in an exemplary embodiment, the present invention provides a data entry landing page for back-up data entry using a corroborator. A second data entry user, a corroborator, may be added for quality control. Once the data entry user has entered the information and the status is complete for the data entry user, the status indicator will be set to incomplete for a second data entry user, for example, a quality control (“QC”) user. The second data entry user may provide backup entry or verification. The data point is sent to the data points column of the landing page for the second data entry user selected to perform this function. Upon entry of data as a backup corroborator, the inventive system automatically assesses whether the data entered by a first data entry user is the same as the data entered by the second data entry user, i.e. the QC user. If the data entered is not the same, the entry user is notified that concordance has not been achieved and an additional corroborator may be added. If the data entered is the same, concordance is achieved 1575. The data entry user may either commit the information to the database or request verification. Once the entry is committed to the database, the audit trail is updated and the status indicator on the data point is set to complete and the data point is removed from data point list 1530. The system saves the data entry history which includes a listing of all prior data entries having the username and the data entered. This also applies to the other data entry stages, such as first entry, verification and flagged data points.

Approval Interface

Similar to the Source capture Interface, an Investigator logs into the inventive system and selects the study and configuration environment they would like to work on. This user interface is for the unique role with the main function for reviewing and electronically signing fully finalized versions of the completed CRF pages and retaining for their records. This user is ultimately responsible for the conduct of clinical trials at the hospital or clinic. This typically is done by the investigator or doctor, a professional at the medical site.

In the workflow currently used in the industry, Investigators review and sign data on CRF documents multiple times when there are edits to the originally submitted answers. In the present invention, only complete and finalized CRF pages are presented to the Investigator for review and signature. Using the preferred inventive workflow disclosed, snippet creation is performed by users who are downstream of the medical site. Therefore, data is fully cleaned and finally entered into a database as the next step after the medical site users provide SC. Once data is entered into the final database, Investigators can review and sign off on data after any steps of data cleaning have been resolved. This is a major departure from current practice and an inventive system and method to enable such a workflow.

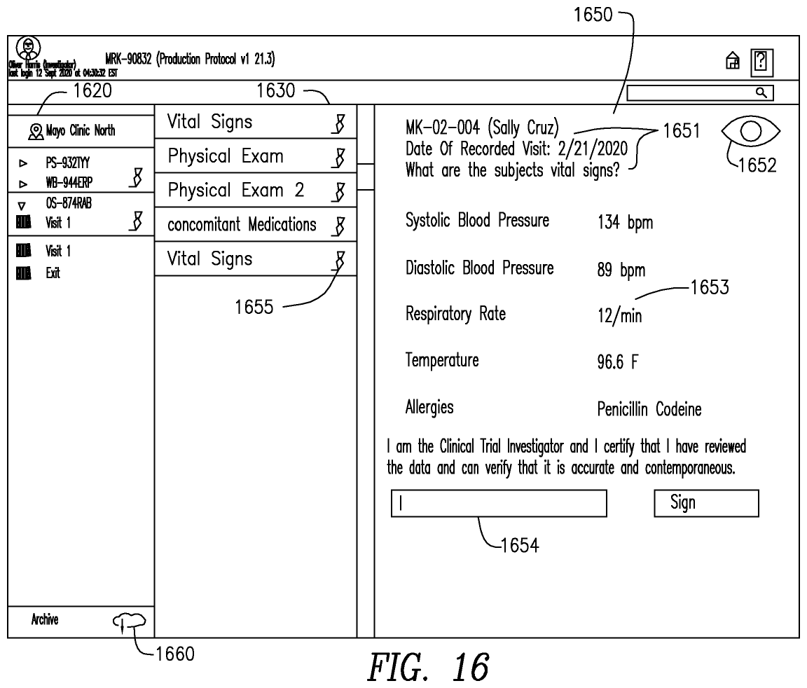

Referring to FIG. 16, in an exemplary embodiment, the present invention provides an Investigator's landing page 1600. Landing page 1600 includes patients column 1620, forms column 1630 and forms detail pane 1650. Investigator landing page 1600 differs from the source capture interface landing page such that the investigator page lists completed packages for the Investigator to review and confirm/approve. These packages are at the end of the process, for example, after data entry and other cleaning queries have occurred. The inventive system notifies the approval user when the data associated with the package is ready to be reviewed and electronically signed by the investigator with signature ready icon 1655 next to a package. Icon 1655 may appear at any or all of the three package levels (form level with all associated questions, visit level with all associated forms or patient level with all associated visits, for example). Icon 1655 indicates that the data is complete with associated SC, is committed to the database and has no open queries. Patient column 1620 includes the site name which, when selected provides a list of patients, or an archive folder. All of the information may be sorted alphabetically. A list of visits for that patient may be shown to the Investigator next to the patient's names or hidden. When a patient visit is selected, the forms associated with the selected visit are displayed in forms column 1630. The selected form is then displayed in form detail pane 1650 which may include a header 1651, review icon 1652, question and answer pairs 1653 and an electronic signature section 1654. Form header 1561 may include the patient study ID, date of visit and form name, for example. Review icon 1652 allows the investigator to view the original SC to verify the source of the snippet question-answer pairs. The SC may be viewed alone, or in a split screen to allow the user to see both the SC and the form at the same time. The investigator may also view the SC in native view or form view. Question-answer pair snippets 1653 provide the full text of the question as written in the CRF and the answer committed to the database by the data entry user, for example. Clicking icon 1655 opens the electronic signature field 1654 at the appropriate package level in form detail pane 1650. Electronic signature section 1654 incudes affidavit text indicating that the electronic signature guarantees that the investigator has reviewed the data and the SC and guarantees the data is correct. Once the e-signature button is clicked, the e-signature is applied to the package, the form detail pane is closed, and the signed content is moved to archive folder 1660 in patients column 1620. It is a regulatory requirement that doctors save clinical trial records for themselves for 15 years. At any time, the doctor can download the full record of all of their patients (in pdf or simple text, for example) by accessing archive 1660.

Robotic Process Automation

An embodiment of the present invention uses Robotic Process Automation (RPA) to automate elements of existing source capture and source document verification (SDV) workflows. This includes automating the processes of accessing the EMR; locating the correct EMR document or data; extracting the EMR data in the form of both screenshots of the electronic source document and extraction of discrete data through automated copying and pasting and through directly reading data from either Windows forms (for native Windows EMR applications) or HTML Document Object Model (for web EMR applications); redacting personal identifying information (PH) through PII pattern matching, OCR, and machine learning; and transmitting the redacted SC and SDV data to centralized servers for storage, collation, analysis, and utilization.

The RPA systems of the present invention are developed for each electronic medical record system used in clinical facilities participating in clinical trials by mapping and automating the user interactions necessary to access patient records, confirm patient identities, and locate relevant clinical trial data within these EMR systems for extraction. The tailored RPA solutions are then deployed to automatically extract SC and SDV data required for a given clinical trial and orchestrated through interactions with a centralized web service (API) that assigns automated RPA data extraction tasks for given study subjects and data fields. Based on the secure web service task-assignment, the automated RPA systems will access EMR records and extract the data assigned through the tasks. (The RPA systems run either remotely or at the client site depending on the networking, data, and legal restrictions). RPA-web service communications are secured through end-to-end TLS encryption securely established over insecure channels using industry-standards such as TLS, Diffie-Helman key exchange, and AES encryption. The RPA system(s) cache extracted data locally in secure, encrypted containers using 256-bit+ AES encryption for all data at rest. The RPA system(s) maintain appropriate credentials for the EMR systems they interact with which are provided by the clinical sites participating in the clinical trials and are stored securely using 256-bit+ AES encryption. The redaction process may occur on the client-side or the server-side. The client-side redaction process ensures that all PII extracted from EMRs is removed from SDV and SC data prior to transmission to the web service, ensuring the web service never processes PII. PII is never persisted to long-term storage on the RPA system(s) and is only maintained in ephemeral memory until redaction is completed.

After transmission to the web service, the RPA-extracted SC and SDV data is validated by human reviewers to ensure no PII is present and the data is accurate. Patient identity is validated through OCR checks on SDV screenshots prior to redaction, checks against discrete EMR data extracted by the RPA, and validation of the procedure to access the data in terms of the user navigation process taken (through automation) by the RPA system to access the data. These validations ensure that the redacted data with no PII is assigned to the correct study subject. If a subject cannot be located by the RPA, or if these validations fail, errors are raised to human operators who can intervene to address the issues, locate the correct data, and address any errors in the RPA process.

For example, a nurse or research coordinator at a clinical site may log into the system of the present invention and select a clinical trial and a specific study subject within that trial. They could further drill down into a specific clinical trial patient visit. Upon doing so, they would be presented with a series of forms that require completion for the selected visit, as well as a button to “automatically complete the selected form(s)”. Selecting the automation button initiates the RPA process. The RPA process would be aware of the relevant EMR pages based on the nurse's prior selection of the study subject, desired visit date, and forms. The RPA in the present invention may then automatically proceed with the following steps, for example:
Opening the EMR software, if it wasn't open already;
Logging into the EMR system on behalf of the nurse/user (if they weren't already logged in), using securely stored credentials;
Automatically “clicking” the correct navigation links of the EMR system and filling out the appropriate navigation forms (e.g. patient search form) to reach the desired information for the correct study subject's record and visit date;
Capturing screenshots of the relevant page(s) containing the answers to the questions on the CRF;
Automatically “drawing” markings on the jpeg or other image files of the screen shots corresponding to the areas that contain answers to individual questions on the CRF in order to create a snippet (a cropped portion of the EMR page screenshot representing an answer to a specific CRF question);
Permanently redacting areas with PII using OCR. Either a client-side OCR or server-side OCR may be used, a configuration toggle may be provided for the user to select the preferred method. Redaction on the client side is performed before the screenshot capture leaves the RPA system by using OCR and a mapping of discrete text data read from the EMR page by the RPA system in order to identify the areas of the screen shot containing the PII. The advantage to client-side processing is that there is zero transfer of PII because any sensitive data is immediately redacted at the point of source capture. On the server side, a screenshot is transmitted to an encrypted fashion to a server where OCR is applied, and redaction occurs before the redacted screenshot is sent back to the client. Redaction can occur within a Google Chrome addon to the user's computer for example. At the server, for example Amazon Wed Service (AWS), the OCR and redaction uses only ethereal memory (RAM) so there is no permanent storage of PII. The server-side processing may be the faster of the two options, however client-side redaction may be the only option for performing redaction where the data privacy rules of the country prohibit the transfer of PII from local citizens to servers outside their borders. In an effort to increase efficiency and speed, a queuing system may be provided where site users may gather a number of screenshots in a session and then continue performing other activities within the system while OCR and redaction processing proceeds in the background. Eventually as screenshots complete processing, the user is notified when screenshots are ready for further interaction.

Using OCR and a mapping of discrete text data read from the EMR page by the RPA system to ascertain the alphanumeric data represented by the image and identify areas of the images containing answers to CRF questions in order to offer suggestions for data entry;
Combining and associating the snippet with the screenshot of the EMR page, the question from the CRF, the underlying alphanumeric data represented in the snippet, and the full audit trail of actions (date/time/user) to formulate an Electronic Data Document (EDD);
Transmitting the EDD (with PII redacted) to the server over a secure network;
Initiating other processes that have already been described to enable human review of the EDD information and final data entry and/or quality check;
Automatically “clicking” the logout button on the EMR system for the nurse/user and providing the nurse/user with a report summarizing the actions taken and the resulting EDD for review, commenting and editing.

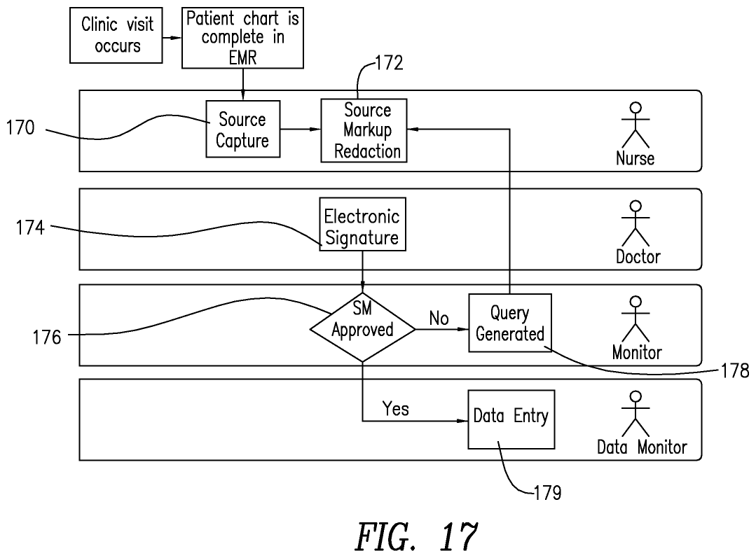

Example Workflow 1 as Shown in FIG. 17
Step 1—The nurse logs into an EMR and navigates to a patient's visit information. The nurse then opens the system of the present invention which presents a list of CRF questions that require a response. The nurse first performs SC, 170, of any pages in the EMR that contain information relevant to the clinical trial questions. The SC images can be organized in a hierarchy by visit date for the study subject, for example. The nurse then begins the process of markup and redaction, 172, if needed such as the patient's name. Any questions where a source document is not necessary would be input as direct entry, while questions requiring source document evidence would require only mouse-driven source markup. During the process of marking up the source document type questions, the system would automatically regroup questions according to their markup on particular SC pages. (A nurse may also manually regroup questions through a dedicated UI).

During source markup, 172, the system will intelligently re-organize the CRF questions to suit the evidence present on any given source capture. If a given source capture only contains 4 out of 7 answers of a 7 question CRF page, the system will re-paginate the CRF to reflect only the 4 answerable questions, and move the remaining 3 questions to a new page that it will auto-label (i.e., the page name ‘Vital Signs’ would become ‘Vital Signs 2’ and so on). Since the screen area captured within the EMR system will remain roughly the same from visit to visit, the system will remember the grouping for future instances. Upon completing all questions, the source markup process ends, and the nurse's work is done.
Step 2— Completion of work by the nurse triggers a notification for the Investigator (doctor) to log into the system. The doctor can see the markup, redactions, and data entries performed by the nurse and edit anything. Finally, when the doctor is satisfied, the system allows the doctor to provide an electronic signature 174. The Investigator's work is done.
Step 3—Sign off by the Investigator triggers a notification for the monitor that a package of visit data is ready for them. The monitor logs into the system and opens the SC but can only see the submitted responses to the non-source document questions and the questions with corresponding snippets. A monitor can use the systems ‘periscope’ view to see the context surrounding any snippet but is unable to see redacted sections of the SC. The monitor then systematically accepts 176 or rejects each response. If the response is rejected, the system opens a dialogue to enable the monitor to place a query 178 on the response, either in the form of free text and/or to choose from a selection of categories. Once all the responses from the site have been addressed, the monitor's work is done. Data that has been accepted by the monitor is considered ‘locked’ and should appear visibly designated as such in the user interface. The site cannot change locked data unless the monitor manually unlocks it by rejecting the data point and placing a query.
Step 4—Any rejected responses from the monitor trigger a notification for the appropriate nurse to view the data point in question. The nurse then logs into the system to see a list of ‘open queries’ with links directly to the relevant SC pages. The nurse can adjust the markup and/or use a dialogue box within the system to respond to the monitor with free text. The nurse's query responses are then re-queued for the monitor to review and either accept (close query) or reject again (re-query) and Step 3 repeats.
Step 5—Any accepted responses from the monitor triggers a notification for the data management personnel to perform data entry (DE) 179 of the responses to all questions that require source document evidence. The system provides an interface to systematically go through each response and transcribe the response. In some cases, a double data entry workflow can be deployed where the data is entered twice (by either the same or different users), with the system flagging discrepancies. The system also allows data managers to code data at this point, by allowing them to select from a menu of pre-determined options. The coding functionality can be performed by the same user who does data entry or a separate user who is specifically designated for this purpose with a workbench and queue of data points that are ready for coding.
Step 6— The system can be used to output data in the form of a CSV file with some parameters enabling a user to map fields to a client specification for purposes of labeling, integration, etc.

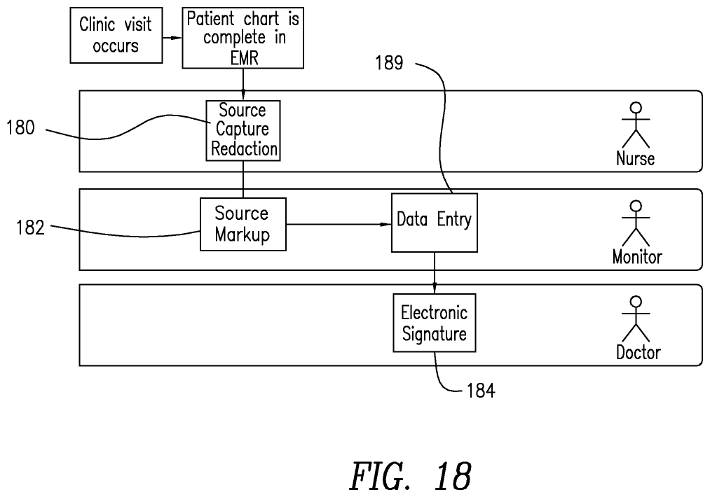

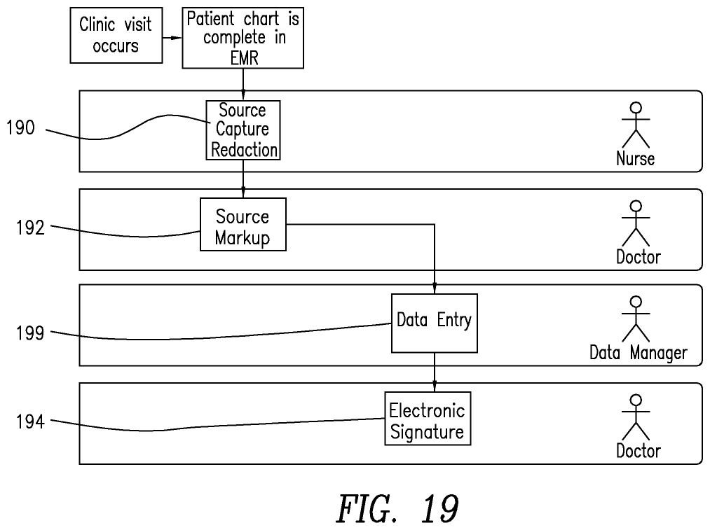

Example Workflow 2 as shown in FIGS. 18 and 19

The same as workflow 1 above, except the monitor can perform the source markup and subsequent data entry. In detail, the site performs SC and Redaction 180, 190. However, while the site may not be performing source markup, they can still Re-Group questions so that any given SC is associated with at least one question from the CRF. The SC becomes digitally associated with the designated grouping of questions, which is then sent to the monitor as a discrete package. The monitor is provided an interface that allows them to source markup 182, 192, according to the questions grouped and attached to the SC by the nurse. Note that this method largely removes any need for queries (although the functionality still exists). While it is conceivable that the monitor might question the groupings associated to the SC by the nurse, there would seldom be a case where a monitor would query themselves on the source markup they just performed. Lastly, in this workflow, the monitor could immediately perform data entry 189 or pass the process forward to a data manager 199 as described in Example Workflow 1 above. A major difference between workflow 1 and workflow 2 is that the doctor's signature 184, 194 in workflow 2 happens after the monitor has completed all their work on a visit and not before.

Example Workflow 3

The same as workflow 2 except the monitor is able to perform SC 202. In detail, the nurse only needs to respond to questions that do not require source document evidence 201. This then triggers a notification for the monitor to log into an EMR to take screenshots and perform markup 200. This method assumes the monitor has their own access to a hospital EMR system. The monitor then performs data entry 209 and the doctor provides an electronic signature 204.

Improvements to Image Snippet Security

The present invention provides improvements to the embodiments presented above that add additional security and further reduce the risk of inadvertent error by verifying the source of the SC. This verification may be driven by OCR and programming logic, secure and encrypted communication (a “handshake”) between the inventive software and the EMR system, checking active EMR application windows, Inter-Process Communication (IPC) and/or web-based API's. The improvements to the inventive system verify that the SC is legitimate by identifying several key pieces of data that exist on the SC. This information may include the patients name (first and last), the patient's date of birth and/or the patients date of visit to the hospital or clinic.

The use of screen OCR technology allows real-time scanning of the user's screen to detect the presence of keywords relating to the current set of CRF questions and snippets. Using OCR determines or confirms the name of the patient for which snippets are being created is on the user's screen at the time of asking. For example, a snippet session starts, a screen reader is activated and searches for keywords on the user's screen. Monitoring continues until information is found, otherwise the session is deemed to be “unverified.”

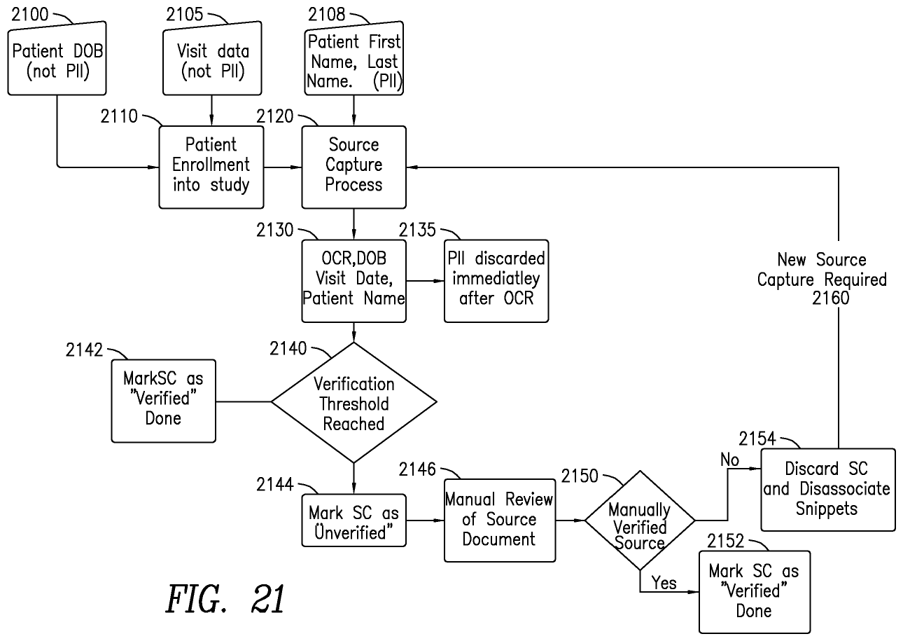

Referring to FIG. 21, an embodiment of the verification process using OCR is shown. Each of the data points above are supplied to the inventive system in different ways. Patient's name 2108 will be supplied to the system at the time of screenshot source capture 2120. The patient's name is considered PII, and as such is only processed in ephemeral memory on the server side and never stored to permanent media. On the client/browser side, patient's name 2108 (the client cache) is purged 2135 at the moment the user logs out of a session. This can be deliberate, or via an automated logout for inactivity based on a certain time threshold. The client cache may also be purged at the moment the web browser software (such as Google Chrome) is quit. Given this architecture, the system never stores the patient's name to permanent media either on the server or on the client side. Patient's date of birth 2100 is supplied by the caregiver staff at the time of enrollment in the clinical trial 2110 and is available to the inventive system. Patient's date of birth 2100, taken in isolation, is not PII. Patient's visit date to the hospital or clinic 2105 is recorded in the EMR and supplied to the inventive system for each visit by the caregiver staff. Visit date 2105 is also not PII.

Once the SC process 2120 has started/occurred, verification driven by OCR 2130. Verification 2140 is based upon what the inventive system is able to find, such as patient DOB 2100, patient visit 2105 and/or patient name 2108. The SC will be assessed as being “verified,” 2142, or “unverified” 2144 and marked with a visually distinct icon so a user, such as a monitor user, can see it. The SC will be designated as “verified” 2142 if all three data points, 2100. 2105, 2108, are positively identified by the system. The SC will be designated as “unverified,” 2144, if one or more data points, 2100. 2105, 2108, are not identified on the system. The degree of stringency in the verification assessment 2140 can be changed as a configuration setting such that in some cases, two out of three data points or certain combinations of data points (such as patient's name 2108 and date of birth 2100) could be considered sufficient to designate a source capture as being “verified,” 2142.

In the event a SC is designated as “unverified,” 2144, it will be marked in the system such that a user, such as the monitor user, will be able to view a list of all “unverified” SCs organized by investigator sites, export the list to a spreadsheet or database, and print the unverified SCs to paper. The user, such as a monitor user, can access the original source media to manually review the SC 2146 and see that the correct SC was taken and manually verify 2150 whether the “unverified” source capture is “acceptable” (verified) 2152 or “rejected” (unverified) 2154 in the inventive system. This allows the user to reconcile the “unverified” SC list for quality assurance measures. Any remaining “unverified” SC 2154 that the monitor had manually reviewed 2150 and double checked by viewing the original EMR and marked as “rejected” 2154 would be flagged for re-capture of the source image 2160, and any snippets arising from the “rejected” SC 2154 would be disassociated from the CRF question and returned to the investigator site to start over and redo from the beginning.

Another verification embodiment includes a device and a process whereby a secure and encrypted communication (a “handshake”) exists between the inventive software creating the SC and the computer window containing the content from the EMR system which the SC originates. This “handshake” between the two software components is preferably a two-way communication whereby an electronic verification takes place each time an SC is created. This electronic verification may be recorded, such that a human-readable log may be generated and added as a component to the EDD described in the embodiments above.

The above described two-way communication verifies that the window in active use (in “focus”) at the time of SC creation is, in fact, the EMR system. In addition, once a two-way communication is created in a secure and encrypted fashion, the EMR may be requested to send data, preferably from an Application Programming Interface (“API”) for the EMR system, that verifies that the window in “focus” is the medical record for the correct patient and/or for the correct visit to the doctor's office.

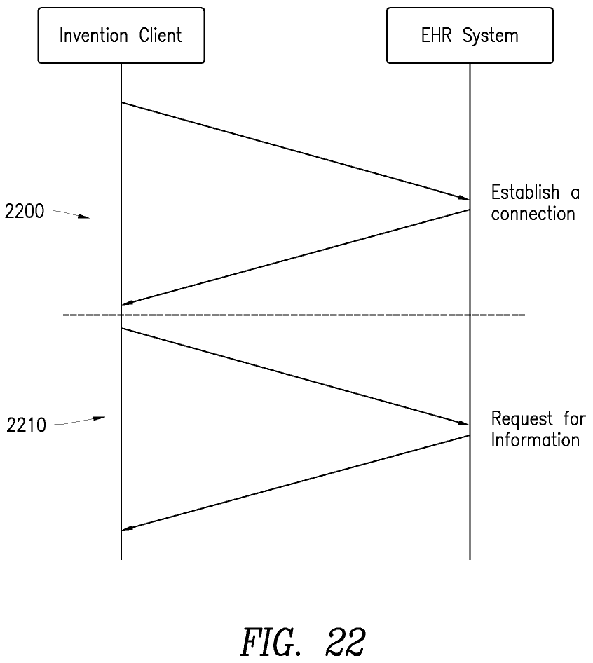

Referring to FIG. 22, an embodiment of this improvement to the system and method described above is shown. The verification of the source of a snippet at the time of capture is a two-step process. The first step, 2200, a “handshake,” occurs when a connection is established and verified to be the EMR application/window providing the source of the snippet or SC. Once the source is validated, the second step, 2210, provides means for the EMR system to confirm it is displaying information about a specific patient and/or a specific visit of said patient. Screen Optical Recognition (OCR), checking active EMR application windows, Inter-Process Communication (IPC) and web-based API's can be used to verify that a current EMR record, for which snippets are being created, matches that of the expected subject.

Another verification embodiment checks active EMR application windows and analyzes the open windows on the user's operating system to yield information about whether a known EMR software application has an active window currently on display. If the window has a title or metadata that provides further insight to its current contents that matches a keyword, the SC is verified. Information such as the name of the patient or an ID and DOB may be used. If no information is available, or the information does not match the keywords queried, the snippet is unverified. For example, when a SC session starts, the inventive software detects the operating system windows. The available meta data is queried for keywords. The presence, or absence, of expected information determines verification.

Another verification embodiment provides uses Inter-Process Communication (IPC) which allows different processes (applications) to share or exchange information concurrently providing the processes involved are all employing a given set of protocols, i.e. they are all listening for messages and expecting to respond as necessary. Through the use of either pipes (unidirectional data channels) or sockets (endpoints for sending or receiving data) encrypted information can be transferred following the secure exchange of cryptographic keys (or handshake) in which both the inventive software and the computer window containing content from the EMR system identify themselves and verify the keys they have been provided. Once authentication is established, messages can be transferred between the two software components to ascertain whether the EMR is displaying information relating to a specific subject. IPC requires the cooperation of the EMR applications which implement software protocols defined by the inventive system. These protocols do not request patient information but only ask that provided information about a particular subject, for example, a name or ID, be confirmed to be the information the application window is displaying at the time of query. For example, when a SC session starts, the inventive system requests a connection with the EMR software via the operating system. Once the connection is established, the EMR software is asked to confirm whether information about a particular patient is currently displayed. The response, or lack thereof, determines verification.

Another verification embodiment uses a web-based API which allows the user, such as an Investigator user, to grant the inventive system to request information directly from the EMR's web service on their behalf, yielding information not only on what is being displayed, but also verifying the user who is accessing it. Using standard secure internet protocols, such as OAuth, the inventive system is granted a token which is sent with requests for information to the EMR API while the SC are being created. The EMR system confirms the patient record currently displayed. This confirmation may also include more detailed non-sensitive patient information such as the type of record being viewed by the user, giving greater veracity to SC data. An authorization token of this kind persists until it is revoked. Therefore, the authorization token may be reused with each new SC event without repeating the authorization steps. For example, the inventive system authorizes with the EMR web service and a token is stored for later use. Once a SC session starts, an API query is made to the EMR software web service to request confirmation that information about a particular patient is currently displayed. The response determines verification of the data source.

The present improvements can provide additional functionality to the inventive system and method that adds further value. The improvements can enable verification between the inventive system and the SC/EMR to ensure the correct patient information is being used.

General

The present invention can take the form of an entirely hardware embodiment, an entirely software embodiment, or an embodiment containing both hardware and software elements. In an exemplary embodiment, the present invention is implemented in software, which includes but is not limited to firmware, resident software, and microcode. Any conventional EMR system can be used as a source of SC, such as Cerner, Epic Systems and Allscripts. Preferably, the systems and methods of the present invention are implemented in a client/server network connected via the Internet.

The systems and methods of the present invention will also preferably use data security, encryption, and data capture and transfer protocols that will enhance patient privacy and security and add desirable authentication and verification features. Preferably, all data will be transferred over SSL connections (also known as HTTPS). Preferably, the systems and methods of the invention will use data encryption (public/private key pair) to protect patient medical records represented in SD media, and data encryption will protect both data and media while they are stored and while they are being transferred, ensuring that only the intended recipients are able to access/view them. Preferably, every user must be authenticated on the system by logging in with their private credentials. Preferably, during each interaction with the server, the server confirms the authenticity of the request for interaction by authentication tokens issued by the server. Preferably, the system will require the users to change their passwords periodically. Preferably, users will only have access to the functionality assigned to them by the system administrator. Preferably, information such as patient or subject ID, data capture date, and other necessary identifying information is embedded in the SC and SD image itself as well as included in metadata, and accessible to qualified users and viewers of the EDD.

In another preferred embodiment, no media or data is saved to any local machine or device, either by the machine or device as it is created or by the monitor/Sponsor or Investigator when viewing it. Rather, data is captured directly from the screen output by the inventive software and is not handled by the native Operating System, which might write that data to disk, even if only as temporarily cached files. Any additional image processing that may be required, such as file compression for storage, is handled by servers away from the local machine or device. Preferably, the computer systems and programs used in embodiments of the invention do not save the EDD or other files created incident to the operation of the invention as a file that can be recalled at a later time. SC can be obtained from any EMR software running on the same machine as the inventive software, such that the EMR software displays its information on the same screen(s) as are accessible by software implementing the invention. Additional digital media imported by the invention from any external source, such as photographs of paper documents or medical scans, are treated in the same manner as SC once loaded.

Furthermore, the present invention can take the form of a computer program product or products accessible from a computer-usable or computer-readable medium providing program code for use by or in connection with a computer system or any instruction execution system. The computer program product includes the instructions that implement the method of the present invention. A computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk, and an optical disk. Current examples of optical disks include compact disk-read only memory (CD-ROM), compact disk-read/write (CD-R/W), and DVD.

A computer system suitable for storing and/or executing program code includes at least one processor coupled directly or indirectly to memory elements through a system bus. The memory elements include local memory employed during actual execution of the program code, bulk storage, and cache memories that provide temporary storage of at least some program code to reduce the number of times code is retrieved from bulk storage during execution. Input/output (I/O) devices (including but not limited to keyboards, displays, pointing devices, etc.) can be coupled to the computer system either directly or through intervening I/O controllers. Network adapters may also be coupled to the computer system in order to enable the computer system to become coupled to other computer systems or remote printers or storage devices through intervening private or public networks. Modems, cable modems, and Ethernet cards are just a few of the currently available types of network adapters. The computer system can also include an operating system and a computer filesystem.

It is to be understood that the above description and examples are intended to be illustrative and not restrictive. Many embodiments will be apparent to those of skill in the art upon reading the above description and examples. The scope of the invention should, therefore, be determined not with reference to the above description and examples but should instead be determined with reference to the appended claims, along with the full scope of equivalents to which such claims are entitled. The disclosures of all articles and references, including patent applications and publications, are incorporated herein by reference for all purposes.

1. A method for electronic verification of a source for a source capture in a clinical trial workflow system, the method comprising:
opening an EMR system;
logging into the EMR system on behalf of a user using securely stored credentials;
automatically selecting correct navigation links of the EMR system and filling out the appropriate navigation forms to reach an EMR page with desired information for a correct study subject's record and visit date;
capturing one or more screenshots of one or more relevant pages containing the desired information for answering one or more questions on a case report form (CRF); and
verifying that the one or more captured screenshots is of the correct study subject by using one or more data points for verification.

2. The method as recited in claim 1, wherein the verifying includes:
real-time scanning by the clinical trial system of the one or more screenshot captures to detect the presence of keywords of the one or more data points; and
assessing if the one or more captured screenshots is verified or unverified, wherein the one or more captured screenshots is labeled verified if a defined number of the one or more data points is positively identified in the one or more captured screenshots by the clinical trial system, wherein the one or more captured screenshots is labeled unverified if the defined number of the one or more data points are not positively identified by the clinical trial system.

3. The method as recited in claim 2, wherein the data points include a study subject's first and last name, a study subject's date of birth and a study subject's visit date.

4. The method as recited in claim 2, further comprising a manual review of the one or more captured screenshots labeled unverified, wherein the user manually reviews the source of the one or more captured screenshots to determine if a correct study subject's data was used, if the correct study subject's data was used in the one or more captured screenshots, the one or more captured screenshots is accepted and labeled verified, wherein if an incorrect study subject's data was used in the one or more captured screenshots, the one or more captured screenshots is rejected and discarded.

5. The method as recited in claim 2, wherein the defined number of data points to be positively identified for verification assessment is set in a configuration setting.

6. The method as recited in claim 4, wherein if the one or more captured screenshots is rejected and discarded, the one or more captured screenshots and related snippets are disassociated from questions on the CRF and returned to an investigator user to start over.

7. The method as recited in claim 1, wherein the electronic verification includes a secure and encrypted two-way communication comprising:
a handshake when a connection is established and verified to be the EMR system providing the source for the one or more captured screenshots; and
once the source is verified, requesting the EMR system to confirm it is displaying information about the correct study subject.

8. The method as recited in claim 7, wherein the electronic verification takes place each time a screenshot is captured.

9. The method as recited in claim 7, wherein the electronic verification is recorded, such that a human-readable log is generated.

10. The method as recited in claim 7, wherein the EMR system confirms it is displaying information about the correct study subject using screen optical recognition, inter-process communication or web-based API's.
